(19) United States
(12) Patent Application Publication
(10) Pub. No.: US 2023/0009265 A1
(43) Pub. Date: Jan. 12, 2023

(54) MANAGEMENT OF DOSE RECOMMENDATIONS

(71) Applicant: Medtronic MiniMed, Inc., Northridge, CA (US)

(72) Inventors: Benyamin Grosman, Winnetka, CA (US); Eric S. Cohen, San Diego, CA (US); Pratik J. Agrawal, Porter Ranch, CA (US); Ellis Garai, Woodland Hills, CA (US); Madison B. Smith, San Diego, CA (US); Haripriyan K. Hampapuram, Carlsbad, CA (US); Yuxiang Zhong, Arcadia, CA (US)

(21) Appl. No.: 17/367,763

(22) Filed: Jul. 6, 2021

(51) Int. Cl. G16H 20/17; A61B 5/00

(52) U.S. Cl. CPC G16H 20/17; A61B 5/742; A61B 5/4839

(57) ABSTRACT

Disclosed herein are techniques related to managing dose recommendations in medicine administering systems. The techniques may involve obtaining a recommendation for a dose of medicine to be delivered to a patient and determining that the recommendation is outdated based on detecting one or more intervening events occurring after generation of the recommendation but before any delivery of medicine in accordance with the recommendation. The techniques may further involve implementing one or more safety features for avoiding delivery of the dose of medicine to the patient.

### FIELD

The present disclosure relates to medicine administering systems and, more particularly, to management of dose recommendations.

### BACKGROUND

Diabetes mellitus (“diabetes”) is a metabolic disease that affects the regulation of glucose by insulin. Diabetes affects hundreds of millions of people and is among the leading causes of death globally. Diabetes has been categorized into three types: type 1, type 2, and gestational diabetes. Type 1 diabetes is associated with the body's failure to produce insulin. Type 2 diabetes is associated with the body's failure to produce sufficient amounts of insulin. Gestational diabetes is associated with insulin-blocking hormones that are produced during pregnancy. Gestational diabetes often resolves after pregnancy; however, in some cases, gestational diabetes develops into type 2 diabetes.

Treatment for various diseases and medical conditions, such as diabetes, typically involves administering doses of medicine. When administering a liquid medicine by injection, for example, an appropriate dosage may be set in a medicine delivery device (e.g., a syringe, a medicine delivery pen, or a pump) and dispensed therefrom. Regardless of the particular delivery device utilized for administering medicine, accurately tracking the medicine dosed can help avoid over-delivery and under-delivery. To this end, some medicine administration systems include software applications that assist users with medicine administration and tracking, thereby helping users manage their diseases and/or medical conditions.

### SUMMARY

To the extent consistent, any of the aspects and features detailed herein can be utilized with any of the other aspects and features detailed herein in any suitable combination. Provided in accordance with aspects of the present disclosure is a system including one or more processors and one or more processor-readable storage media. The one or more processor-readable storage media store instructions which, when executed by the one or more processors, cause performance of obtaining a recommendation for a dose of medicine to be delivered to a patient and determining that the recommendation is outdated based on detecting one or more intervening events occurring after generation of the recommendation but before any delivery of medicine in accordance with the recommendation. The instructions, when executed by the one or more processors also cause performance of implementing one or more safety features for avoiding delivery of the dose of medicine to the patient.

In some aspects of the present disclosure, implementing the one or more safety features includes causing presentation, via a user interface of a medicine delivery device, information indicating that the recommendation is unreliable.

In some aspects of the present disclosure, the medicine delivery device is a medicine injection device.

In some aspects of the present disclosure, the user interface includes one or more light-emitting diodes (LEDs).

In some aspects of the present disclosure, causing presentation of the information includes causing one or more LEDs to illuminate a particular color.

In some aspects of the present disclosure, the information indicates that the recommendation is unreliable due to the recommendation being outdated.

In some aspects of the present disclosure, the information indicates that the recommendation is unreliable due to passage of a threshold period of time since communication between the medicine delivery device and a computing device.

In some aspects of the present disclosure, implementing the one or more safety features includes causing activation of a mechanical lock-out mechanism configured to prevent delivery of medicine from a medicine delivery device.

In some aspects of the present disclosure, detecting the one or more intervening events includes determining passage of a threshold period of time since generation of the recommendation.

In some aspects of the present disclosure, detecting the one or more intervening events includes obtaining a superseding recommendation for an updated dose of medicine to be delivered to the patient.

In some aspects of the present disclosure, the one or more processor-readable storage media further store instructions which, when executed by the one or more processors, cause performance of after obtaining the recommendation, determining that the recommendation is up-to-date based on an absence of intervening events occurring after generation of the recommendation but before any delivery of medicine in accordance with the recommendation and causing presentation, via a user interface of a medicine delivery device, information indicating that the recommendation is reliable.

Also provided in accordance with aspects of the present disclosure is a processor-implemented method. The method includes obtaining a recommendation for a dose of medicine to be delivered to a patient and determining that the recommendation is outdated based on detecting one or more intervening events occurring after generation of the recommendation but before any delivery of medicine in accordance with the recommendation. The method also includes implementing one or more safety features for avoiding delivery of the dose of medicine to the patient.

In some aspects of the present disclosure, implementing the one or more safety features includes causing presentation, via a user interface of a medicine delivery device, information indicating that the recommendation is unreliable.

In some aspects of the present disclosure, the user interface includes one or more light-emitting diodes (LEDs), and causing presentation of the information includes causing one or more LEDs to illuminate a particular color.

In some aspects of the present disclosure, the information indicates that the recommendation is unreliable due to the recommendation being outdated.

In some aspects of the present disclosure, the information indicates that the recommendation is unreliable due to passage of a threshold period of time since communication between the medicine delivery device and a computing device.

In some aspects of the present disclosure, implementing the one or more safety features includes causing activation of a mechanical lock-out mechanism configured to prevent delivery of medicine from a medicine delivery device.

In some aspects of the present disclosure, detecting the one or more intervening events includes determining passage of a threshold period of time since generation of the recommendation.

In some aspects of the present disclosure, detecting the one or more intervening events includes obtaining a superseding recommendation for an updated dose of medicine to be delivered to the patient.

Also provided in accordance with aspects of the present disclosure is one or more non-transitory processor-readable storage media storing instructions which, when executed by one or more processors, cause performance of obtaining a recommendation for a dose of medicine to be delivered to a patient and determining that the recommendation is outdated based on detecting one or more intervening events occurring after generation of the recommendation but before any delivery of medicine in accordance with the recommendation. The instructions, when executed by the one or more processors, also cause performance of implementing one or more safety features for avoiding delivery of the dose of medicine to the patient.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a schematic illustration of an example medicine administration and tracking system provided in accordance with the present disclosure including a medicine injection pen, a computing device, and, in aspects, a sensor device and/or a data processing system;

FIG. 1B is a block diagram of the medicine injection pen of the system of FIG. 1A;

FIG. 1C is a block diagram of the computing device of the system of FIG. 1A; and

FIG. 2 is a flowchart illustrating an example approach for managing dose recommendations in accordance with the present disclosure.

### DETAILED DESCRIPTION

The present disclosure relates to management of dose recommendations for medicine administration. Treatment for various diseases and medical conditions, such as diabetes, typically involves administering doses of a fluid medication. For example, when administering a fluid medication, the appropriate dose amount may be set in a medicine-administering device (e.g., a syringe, a pen, or a medicine infusion pump) and dispensed therefrom. The medicine-administering device may be part of a medicine administering system that also includes one or more sensor devices (e.g., a wearable continuous glucose monitor) that monitor a person's health metrics (e.g., glucose levels). Based on information from these devices (e.g., information concerning a previously administered dose of medicine and/or information concerning glucose levels), a dose recommendation may be provided to a person (e.g., a patient or some other user).

However, a person's physiological condition tends to change with time, particularly as the person performs different activities (e.g., exercise, eating, sleeping, etc.). As such, a dose recommendation that is not promptly acted upon may become outdated. Moreover, administering medicine based on an out-of-date recommendation may result in underdosing or overdosing of medication.

To avoid the aforementioned shortcomings, disclosed herein are techniques related to management of dose recommendations to help avoid administering of medicine doses based on an out-of-date dose recommendation. For example, a personal computing device (e.g., a smartphone) and/or a medicine-administering device may be used to determine if a dose recommendation is outdated. This determination may be based on detecting any number of a variety of intervening events (e.g., passing of a threshold amount of time, user activity, various user inputs, and/or a change in dose calculation) that occur after generation of the dose recommendation but before the dose recommendation is acted upon if at all. Various safety features may also be implemented to prevent the person from acting on an out-of-date dose recommendation. For example, in response to a determination that a dose recommendation is out-of-date, a medicine administration operation of a medicine-administering device may be prevented and/or various alerts may be generated to notify the person that the dose recommendation is out-of-date.

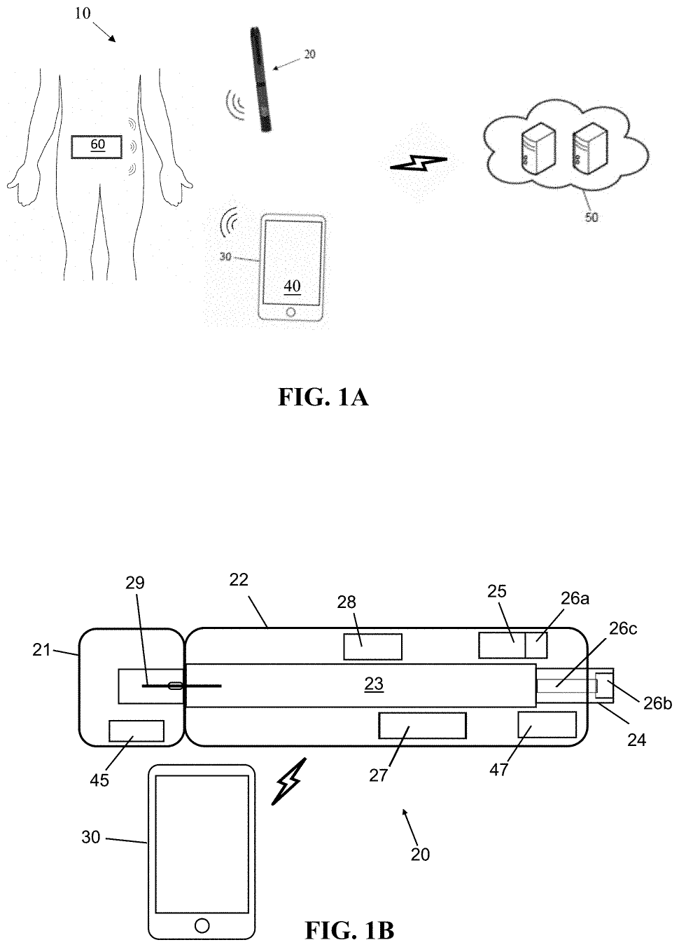

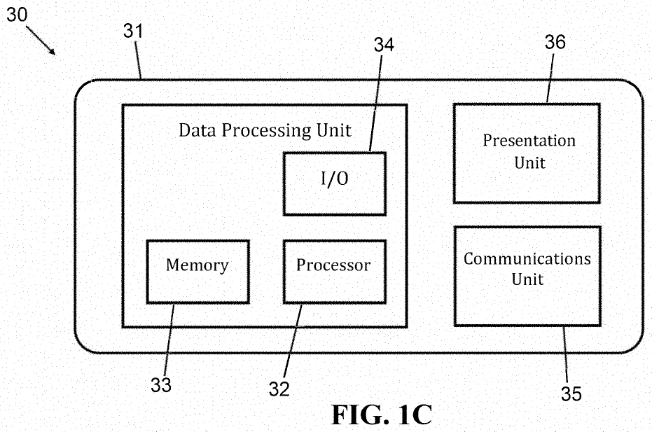

FIG. 1A illustrates an example medicine administration and tracking system 10 provided in accordance with the present disclosure including a medicine injection pen 20 in wireless communication with a computing device 30 running a health management application 40 associated with pen 20 and/or other devices that are part of or connected to system 10. System 10, in aspects, further includes a data processing system 50 and/or a sensor device 60. While the reusable injection pens with replaceable cartridges of the present disclosure are detailed herein configured for use as medicine injection pen 20 of system 10 with respect to diabetes management, it is understood that the reusable injection pens of the present disclosure are also applicable to management of other diseases and medical conditions and/or for use with other medicine administration and tracking systems.

Medicine injection pen 20, described in greater detail below, may be a reusable injection pen configured to removably receive a medicine cartridge, e.g., a cartridge of insulin, for injecting a selected dose of insulin into a patient and recording information concerning the injected dose of insulin, e.g., a dose amount and/or timestamp data associated with the dose.

Computing device 30 is detailed and illustrated herein as a smartphone, although any other suitable computing device may be provided such as, for example, a tablet, a wearable computing device (e.g., a smart watch, smart glasses, etc.), a laptop and/or desktop computer, a smart television, a network-based server computer, etc. Thus, solely to provide a clear example, computing device 30 is hereinafter referenced as smartphone 30.

Smartphone 30 may be paired with pen 20, which may be a prescription-only medical device, although other suitable configurations are also contemplated. In aspects, the pairing of smartphone 30 with pen 20 at least partially unlocks health management application 40 to enable the user to utilize some or all features of health management application 40, e.g., according to the user's prescription. Thus, the act of pairing can unlock and enable the functionality of health management application 40 and/or system 10 (including pen 20), while health management application 40 (and/or system 10) may provide only limited features in the absence of pairing with pen 20.

Health management application 40 of smartphone 30, in aspects, can monitor and/or control functionalities of pen 20. Application 40 may include one or more sets of instructions corresponding to a dose calculator module and/or decision support module that can cause calculation of dose recommendations (e.g., a recommended dose of medicine for the user to administer using pen 20). Health management application 40 may implement a user interface on the hardware of smartphone 30 to allow a user to manage health-related data. For example, health management application 40 can be configured to control some functionalities of pen 20 and/or to provide an interactive user interface to allow a user to manage settings of pen 20 and/or settings for smartphone 30 that can affect the functionality of system 10 (FIG. 1A). Smartphone 30 can additionally or alternatively be used to obtain, process, and/or display contextual data that can be used to relate to the health condition of the user, including the condition for which pen 20 is used to treat. For example, smartphone 30 may be operable to track the location of the user; physical activity of the user including step count, movement distance and/or intensity, estimated calories burned, and/or activity duration; and/or interaction pattern of the user with smartphone 30. In aspects, health management application 40 can aggregate and process the contextual data to generate decision support outputs, e.g., on the user interface, to guide and aid the user in monitoring his/her condition, using pen 20, and/or managing his/her behavior to promote treatment and better health outcomes.

In aspects, system 10 further includes a data processing system 50 in communication with pen 20 and/or smartphone 30. Data processing system 50 can include one or more computing devices in a computer system and/or communication network accessible via the internet, e.g., including servers and/or databases in the cloud. System 10 can additionally or alternatively include sensor device 60 to monitor one or more health metrics and/or physiological parameters of the user. Examples of health metric and physiological parameter data monitored by sensor device 60 include analytes (e.g., glucose), heart rate, blood pressure, user movement, temperature, etc. Sensor device 60 may be a wearable sensor device such as a continuous glucose monitor (CGM) to obtain interstitial glucose measurements that are processed to produce continuous glucose values. For example, the CGM can include suitable electronics (e.g., a processor, a memory, a transceiver, and/or a battery or other suitable power source) for processing, storing, and/or causing display of the continuous glucose values for the user. Such continuous glucose values can be utilized by health management application 40, for example, for displaying health data, in dose calculation and/or decision support, etc. In addition to or in lieu of the dose calculator module and/or decision support module included in health management application 40, pen 20, in aspects, may include a dose calculator module and/or decision support module that can cause calculation of dose recommendations (e.g., a recommended dose of medicine for the user to administer using pen 20) utilizing, for example, glucose measurements provided by the CGM.

With reference to FIG. 1B, pen 20 includes a cap 21 configured to protect a medicine dispensing element (e.g., a needle 29) and a body 22 configured to contain a replaceable medicine cartridge 23, e.g., an insulin cartridge. Pen 20 further includes a dose dispensing mechanism 24 to dispense (e.g., deliver) medicine contained in medicine cartridge 23 out of pen 20 (e.g., through needle 29); a dose setting mechanism 25 to enable the selection and/or setting of a dose of medicine to be dispensed; an operations monitoring mechanism 28 (e.g., including one or more switches, sensors (electrical, optical, acoustic, magnetic, etc.), encoders, etc.) to determine that pen 20 is being operated and/or to monitor the operation of pen 20 (e.g., to determine an amount of medicine set and/or dosed); and an electronics unit 27 that can include a processor, a memory, a transceiver, and a battery or other suitable power source.

In aspects of the present disclosure, pen 20 may optionally include an indicator mechanism 45 that serves to provide the user with an alert or other indication associated with a medicine dose recommendation. As described in greater detail below, the medicine dose recommendation may be provided via the user interface of health management application 40 and/or the user interface of smartphone 30. In aspects, indicator mechanism 45 may include any suitable mechanism for communicating an alert or other indication to the user, examples of which include but are not limited to a light emitting diode (LED), a digital display, an audio output device, etc. While FIG. 1B shows indicator mechanism 45 disposed on cap 21, it should be understood that indicator mechanism 45 (or additional indicator mechanisms), in aspects, may be disposed on body 22 or on any suitable location of pen 20 such that indicator mechanism 45 is readily accessible (e.g., visible and/or audible) to the user.

In aspects, in order to operate pen 20, the user sets, e.g., dials, the amount of a dose using a dose knob 26a of dose setting mechanism 25. For example, the amount of the dose may be adjusted up or down to achieve a desired dose amount prior to administration of the dose by rotating dose knob 26a in an appropriate direction. Once the appropriate dosage has been set, the user applies a force against a dose dispensing button 26b of dose dispensing mechanism 24 to begin dispensing. More specifically, to begin dispensing, the user presses against the portion of dose dispensing button 26b that protrudes from body 22 of pen 20 to thereby drive a driving element 26c, e.g., a drive screw, of dose dispensing mechanism 24 against an abutment, e.g., a piston, of medicine cartridge 23 to dispense an amount of medicine from cartridge 23 through needle 29 into the user in accordance with the dose amount set by dose setting mechanism 25, e.g., dose knob 26a, during setting.

Operations monitoring mechanism 28 of pen 20 may sense movement of a rotating and/or translating driving element 26c of dose dispensing mechanism 24. Operations monitoring mechanism 28 may include one or more switches, sensors, and/or encoders for this purpose. More specifically, any suitable switch(es), sensor(s), and/or encoder(s) may be utilized to sense rotary and/or linear movement. Non-limiting examples of such include rotary and linear encoders, Hall effect and other magnetic-based sensors, linearly variable displacement transducers, optical sensors, etc. With respect to an encoder, for example, the encoder can be configured to sense the rotation of driving element 26c that also translates to dispense medicine; thus, by sensing rotation of driving element 26c, the translational movement of driving element 26c can be readily determined. Movement of the encoder may be represented as data that can be used to determine, by the processor of electronics unit 27 of pen 20, the amount of medicine dosed.

In aspects, the memory of electronics unit 27 of pen 20 can store the amount of a dose along with a timestamp for that dose and/or any other information associated with the dose. In aspects, the transceiver of electronics unit 27 enables pen 20 to transmit the amount of the dose and related information to smartphone 30. In such aspects, once the amount of the dose is transmitted, the dose data and any related information associated with that particular dose may be marked as transmitted. If the dose is not yet transmitted to smartphone 30 such as, for example, because no connection between the pen 20 and smartphone 30 is available, then the dose data and any related information can be transmitted the next time a successful communication link between pen 20 and smartphone 30 is established.

In aspects, health management application 40 can update (e.g., replace, add, remove, etc.) a dose recommendation to ensure that the dose recommendation is up to date and to prevent the user from mistakenly dosing or not dosing based on an out-of-date recommendation. For example, a dose recommendation that is not acted upon by the user within a suitable length of time may become outdated for various reasons such as a user activity (e.g., exercise, eating, sleeping, etc.) performed since generation of the dose recommendation and/or changes in glucose values (e.g., provided by the CGM) on which calculation of the dose recommendation was based.

Dose dispensing mechanism 24 of pen 20 can include a manually powered mechanism, a motorized mechanism, or an assisted mechanism (e.g., a mechanism that operates partly on manual power and partly on motorized power). Regardless of the particular configuration of the dose dispensing mechanism 24, as noted above, when a force (e.g., a manual force, electrically powered motor force, or combinations thereof) is applied to driving element 26c of dose dispensing mechanism 24, driving element 26c provides a force to displace medicine from medicine cartridge 23 according to the set or dialed amount. In aspects, dose dispensing mechanism 24 can be operated such that rotation and/or translation of the driving element, e.g., driving element 26c, is facilitated by a variable tension spring or a variable speed motor to inject the dose over a specific time frame (e.g., 1 s, 5 s, etc.) to help reduce the pain of dosing and/or for other purposes.

FIG. 1C illustrates smartphone 30 of system 10 (FIG. 1A) including a data processing unit 31, a communications unit 35, and a presentation unit 36. Data processing unit 31 includes a processor 32 to process data, a memory 33 in communication with the processor 32 to store data, and an input/output unit (I/O) 34 to interface with other modules, units, and/or devices of smartphone 30 and/or external devices. Processor 32 can include a central processing unit (CPU) or a microcontroller unit (MCU). Memory 33 can include and store processor-executable code, which when executed by processor 32, configures the data processing unit 31 to perform various operations, e.g., such as receiving information, commands, and/or data, processing information and data, and transmitting or providing information/data to another device. In aspects, data processing unit 31 can cause transmission of raw or processed data to data processing system 50 (FIG. 1A). To support various functions of data processing unit 31, memory 33 can store information and data, such as instructions, software, values, images, and other data processed or referenced by processor 32. For example, various types of Random-Access Memory (RAM) devices, Read Only Memory (ROM) devices, Flash Memory devices, and other suitable storage media can be used to implement storage functions of memory 33. I/O 34 of data processing unit 31 enables data processing unit 31 to interface with communications unit 35 to utilize various types of wired or wireless interfaces (e.g., a wireless transmitter/receiver (Tx/Rx)) compatible with typical data communication standards to enable communication between data processing unit 31 and other devices such as pen 20. Examples of typical data communication standards include, but are not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, NFC (Near Field Communication), and parallel interfaces. I/O 34 of data processing unit 31 can also interface with other external interfaces, sources of data storage, and/or visual or audio display devices, etc. to retrieve and transfer data and information that can be processed by processor 32, stored in memory 33, and/or exhibited on an output unit of smartphone 30 and/or an external device. For example, presentation unit 36 of smartphone 30 can be configured to be in data communication with data processing unit 31, e.g., via I/O 34, to provide a visual presentation, an audio presentation, and/or other sensory presentation for implementing the user interface of the health management application 40 (FIG. 1A). In some examples, presentation unit 36 can include various types of screen displays, speakers, or printing interfaces, e.g., including but not limited to, LED, liquid crystal display (LCD) monitor or screen, cathode ray tube (CRT), audio signal transducer apparatuses, and/or toner, liquid inkjet, solid ink, dye sublimation, inkless (e.g., such as thermal or UV) printing apparatuses, etc.

Once smartphone 30 receives the dose data and related information (e.g., which can include time information; dose setting and/or dose dispensing information; and/or other information about pen 20 and/or the environment as it relates to a dosing event), smartphone 30 stores the dose related information, which can be included among a list of doses or dosing events. In aspects, via the user interface of the health management application 40, smartphone 30 allows the user to browse a list of previous doses, to view an estimate of current medicine active in the patient's body (medicine on board, e.g., insulin on board) based on calculations performed by health management application 40. Additionally or alternatively, smartphone 30 may allow the user to utilize a dose calculation module to assist the patient regarding dose setting information (e.g., the size of the next dose(s) to be delivered). For example, the patient may enter carbohydrates to be eaten, and health management application 40 may recommend a dosage based on insulin-on-board information. In aspects, smartphone 30 can also allow the user to manually enter dose data, e.g., boluses, which may be useful if the battery in pen 20 has been depleted or another medicine delivery device, e.g., a syringe, was utilized to dose.

Users expect medicine dose recommendations (e.g., insulin dose recommendations) to be based on accurate dose calculations using data that is current. Outdated dose recommendations may result in overdosing or underdosing of medication. To increase safety and help ensure that an outdated dose recommendation is not acted on by the user, health management application 40 may update dose recommendations and/or remove dose recommendations that are no longer relevant in view of, for example, new data input such as carbohydrates eaten (or to be eaten) and/or current glucose values measured by sensor device 60 (FIG. 1A)).

A dose recommendation provided by health management application 40 may become outdated with the passage of time. During that time, one or more user activities may cause the dose recommendation to become outdated. For example, a user may exercise, eat, and/or sleep subsequent to a dose recommendation such that the dose recommendation becomes outdated due to a change in the user's glucose levels. Even without one or more user activities, the dose recommendation may become outdated over time. For example, glucose levels may gradually fall over time if a user refrains from eating, so a dose recommendation may become outdated due to changes in the glucose levels upon which the dose recommendation was based. Health management application 40 may remove outdated dose recommendations from the user interface of health management application 40 and/or the user interface of smartphone 30.

To increase safety and help ensure that the user is acting on an up-to-date dose recommendation, it is helpful to have safety features to increase the likelihood the user will not act on outdated dose recommendations. Such safety features may include indicator mechanism 45 of FIG. 1B. Indicator mechanism 45 may, in aspects, include one or more LEDs that illuminate to provide the user with information regarding the status of dose recommendations and/or information regarding the communication status between pen 20 and smartphone 30. In aspects, health management application 40 may control the functionality of pen 20 and/or cap 21 or otherwise cooperate with pen 20 and/or cap 21 (e.g., by causing dose recommendation to be communicated to pen 20 and/or cap 21) to cause one or more LEDs of indicator mechanism 45 to illuminate (e.g., a particular color, in a particular pattern, and/or in a particular sequence) to provide such information to the user.

As mentioned above, mechanism 45 may convey information regarding the status of dose recommendations. In aspects, one or more LEDs of indicator mechanism 45 may serve to alert the user that a dose recommendation remains up to date, is outdated, has been updated and is now up to date, or that a dose recommendation has been removed. By way of example, one or more LEDs of indicator mechanism 45 may illuminate the color “green” to indicate to the user that the current or most recently generated dose recommendation provided by health management application 40 is up to date and thus, it is safe for the user to act upon the dose recommendation (e.g., administer a dose of medication). Alternatively, one or more of LEDs of indicator mechanism 45 may illuminate the color “red” to indicate to the user that the current or most recently generated dose recommendation provided by health management application 40 is out of date and may not be safe for the user to act upon (e.g., because a threshold amount of time has passed since generation of the dose recommendation).

As mentioned above, mechanism 45 may convey information regarding the communication status between pen 20 and smartphone 30. In aspects, one or more LEDs of indicator mechanism 45 may serve to alert the user that communication and/or a data network connection between pen 20 and smartphone 30 has not occurred or been established for some period of time and/or since the last administered dose of medication. By way of example, one or more LEDs of indicator mechanism 45 may illuminate the color “green” to indicate to the user that the last communication between pen 20 and smartphone 30 occurred within a threshold period of time (e.g., no more than 5 minutes ago). Alternatively, one or more LEDs of indicator mechanism 45 may illuminate the color “red” to indicate to the user that the last communication between pen 20 and smartphone 30 occurred beyond a threshold period of time (e.g., more than 5 minutes ago).

It should be understood that the colors “green” and “red” are used as examples only and that other illumination colors, symbols, combinations thereof, etc. are contemplated.

To increase safety and help ensure that the user is acting on an up-to-date dose recommendation, pen 20 may include safety features in the form of fail-safes, such as an onboard mechanical lock-out mechanism 47 (FIG. 1B) that prevents the user from over-administering a medication dose and/or that prevents the user from administering a medication dose altogether. In aspects, health management application 40 may control the functionality of pen 20 or otherwise cooperate with pen 20 (e.g., by causing information related to a dose recommendation to be communicated to pen 20) to cause lock-out mechanism 47 to engage one or more components of pen 20 to prevent pen 20 from administering a dose or to serve as a flow-control mechanism for controlling the flow of medication from cartridge 23 so that a dose of medication administered by pen 20 does not exceed a recommended dose. Lock-out mechanism 47 may include a latch (not shown) which, when activated, may releasably engage or contact a suitable mechanical interface (e.g., aperture, recess, protrusion, slot, etc.) of dose dispensing button 26b and/or driving element 26c to prevent or limit movement of dose dispensing button 26b and/or driving of driving element 26c. More specifically, the engagement of the latch with the suitable mechanical interface may serve to prevent translational movement and/or rotation of driving element 26c to dispense a medication dose. Additionally or alternatively, the engagement of the latch with the suitable mechanical interface may serve to prevent movement of dose dispensing button 26b toward body 22 of pen 20 so that a user is prevented from causing translational movement of driving element 26c to dispense a medication dose by pressing against the portion of dose dispensing button 26b that protrudes from body 22 of pen 20. Thus, lock-out mechanism 47 may prevent the user from administering a medication dose based on an outdated dose recommendation at least until the outdated dose recommendation is updated and/or replaced. Additionally or alternatively, lock-out mechanism 47 may allow the user to administer medication in accordance with a recommended dose but prevent further flow of medication from cartridge 23 once the recommended dose is dispensed from cartridge 23 to prevent the user from over-administering medication. Health management application 40, in aspects, may control the functionality of pen 20 or otherwise cooperate with pen 20 (e.g., by causing information related to a dose recommendation to be communicated to pen 20) to override lock-out mechanism 47 in specific circumstances to allow for the user to administer more medication than was calculated for the dose recommendation.

In aspects, notification preferences may be modified for dose recommendations and/or updates to dose recommendations. For example, the user may specify designated time ranges of the day in the settings of health management application 40 during which the user does not wish to receive dose recommendations or updates to dose recommendations such as, for example, overnight, sleeping hours, during work meetings, during mealtime, etc. Alternatively, the user may adjust the frequency with which dose recommendations and/or updates to dose recommendations are provided by health management application 40 (e.g., from every five minutes to every one hour) during specified time ranges. However, depending on the severity of user's physiological condition, notification preferences may be overridden. For example, if the user's glucose level approaches a dangerous level, health management application 40, in aspects, can ignore the user's notification preferences and provide (e.g., push) a dose recommendation to the user.

In aspects, health management application 40 may automatically modify notification settings based on various factors (e.g., the user's environment, the user's compliance or lack of compliance with a dose recommendation, the severity of the user's physiological condition, the day of the week, and/or the time of day). For example, health management application 40 may provide multiple notifications and/or progressively increase the frequency with which such notifications are presented if a dose recommendation is ignored by the user. Additionally or alternatively, successive notifications may involve additional sensory stimuli (e.g., visual, audio, and/or tactile stimuli) and/or progressively increasing strength of such stimuli. For example, an initial notification may not be accompanied by an audio alert, but successive notifications may be accompanied by audio alerts that increase in volume until either the user acts upon the notifications or the associated dose recommendation becomes outdated.

In aspects, health management application 40 may generate a report of the user's compliance with dose recommendations. Typically, users of system 10 comply with dose recommendations (e.g., administer recommended doses in a timely manner), ignore dose recommendations and fail to take any action, or administer amounts of medication that are lower or higher than recommended dosages. For example, despite a dose recommendation to abstain from administering a dose of medicine, a non-compliant user may administer a dose anyway. Health management application 40 can track the user's behavior with respect to compliance with dose recommendations and generate a user compliance assessment report that conveys the effects of non-compliance with a dose recommendation versus compliance with a dose recommendation. The report may be generated daily, weekly, monthly, or however often is suitable to facilitate behavior change in the user.

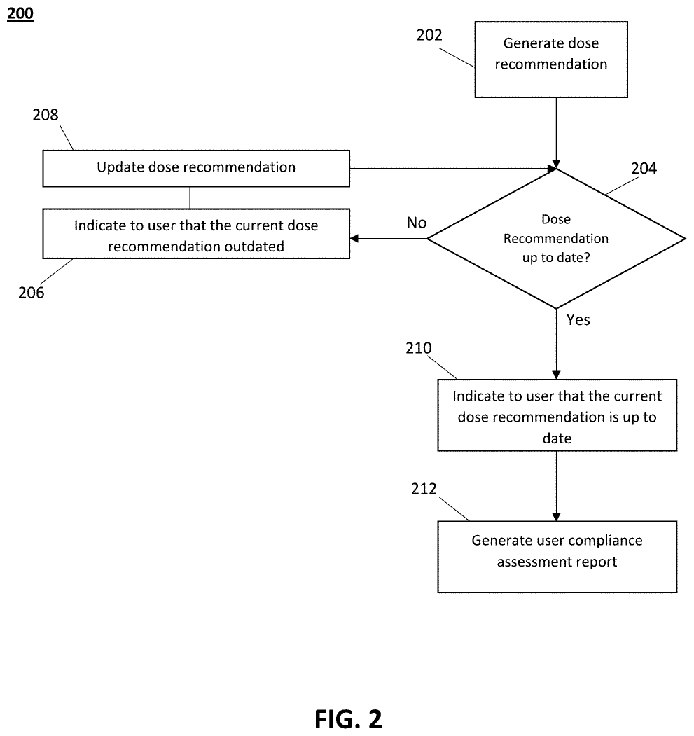

Turning to FIG. 2, also provided in accordance with the present disclosure is an example approach 200 that may be implemented by health management application 40 for providing up-to-date dose recommendations to the user, preventing the user from acting upon out-of-date dose recommendations, and/or generating a report including a user compliance assessment to effect behavior change in the user over time to drive improved compliance with dose recommendations. In aspects, approach 200 may be fully automated.

At block 202, the dose calculator module of health management application 40 of smartphone 30 may cause calculation of a recommended medicine dose and may cause presentation, based on the calculation, of a dose recommendation to the user via the user interface of smartphone 30 for the user to self-administer using pen 20.

At block 204, health management application 40 may periodically or continuously determine if the dose recommendation presented to the user is up to date. This determination may be based on detecting any number of a variety of intervening events that occur after generation of the dose recommendation but before the dose recommendation is acted upon if at all. Example intervening events include, without limitation, the passing of a threshold amount of time, user activity (e.g., exercise, sleep, eating), various user inputs to health management application 40, and/or a change in dose calculation caused by the dose calculation module of health management application 40 based on the user's current blood glucose (e.g., input from the user or directly from sensor device 60 (FIG. 1A)).

If health management application 40 determines that the current dose recommendation is not up-to-date, health management application 40 may provide an indication to the user that the current dose recommendation is out-of-date at block 206 and, in aspects, the health management application 40 may remove or otherwise prevent presentation of the out-of-date dose recommendation from the user interface of smartphone 30. The indication at block 206 may be an alert presented on the user interface of health management application 40 and/or the user interface of smartphone 30. Additionally or alternatively, health management application 40 may control the functionality of pen 20 or otherwise cooperate with pen 20 (e.g., by causing information related to a dose recommendation to be communicated to pen 20) to cause one or more LEDs of indicator mechanism 45 to illuminate to indicate to the user that the current dose recommendation is out-of-date. For example, one or more LEDs of indicator mechanism 45 may be illuminated in the color “red” until the dose recommendation is updated to ensure that the user does not act upon an out-of-date dose recommendation. In aspects of the present disclosure, indicator mechanism 45 may be utilized in combination with or in lieu of lock-out mechanism 47. Additionally or alternatively, health management application 40 may communicate with other applications of smartphone 30 and/or other connected devices to erase, retract, and/or override any notifications (e.g., push notifications) and/or other outputs relating to the outdated dose recommendation.

The dose calculator module of health management application 40, at block 208, may cause recalculation of a recommended medicine dose and may cause updating of the dose recommendation (or replacement of an out-of-date dose recommendation) based on the recalculation. Block 208 may proceed to block 204.

If health management application 40 determines, at block 204, that the current dose recommendation is currently up-to-date, health management application 40 may provide an indication to the user that the current dose recommendation is up-to-date at block 210. This indication may be an alert presented on the user interface of health management application 40 and/or the user interface of smartphone 30. Additionally or alternatively, health management application 40 may control the functionality of pen 20 or otherwise cooperate with pen 20 (e.g., by causing information related to a dose recommendation to be communicated to pen 20) to cause one or more LEDs of indicator mechanism 45 to illuminate to indicate to the user that the current dose recommendation is up-to-date, and/or to provide confirmation (e.g., by modifying, reproducing, refreshing, etc.) that any notifications or outputs from health management application 40 are up-to-date. For example, one or more LEDs of indicator mechanism 45 may be illuminated in the color “green” while the dose recommendation remains up-to-date to ensure the user that he/she is acting on a dose recommendation that is up-to-date. Optionally, when health management application 40 determines that the current dose recommendation is up-to-date, health management application 40 may control the functionality of pen 20 or otherwise cooperate with pen 20 (e.g., by causing information related to a dose recommendation to be communicated to pen 20) to override or disengage lock-out mechanism 47, which may have been previously activated due to an outdated dose recommendation.

At block 212, health management application 40 optionally generates a user compliance assessment report of the user's performance with respect to compliance with dose recommendations. For example, when a determination is made at block 204 by health management application 40 that the current dose recommendation is not up-to-date, system 10 may maintain information related to the cause of the dose recommendation becoming outdated. As an example, the user compliance assessment report may indicate if and/or how often dose recommendations become outdated due to the user failing to take any action within a threshold amount of time following generation of a dose recommendation. Additionally or alternatively, the user compliance assessment report may indicate if and/or how often the user ignored an indication that a dose recommendation was outdated and administered a dose anyway. Even if the dose recommendation is determined to be up-to-date at block 204, system 10 may keep track of whether the user complied with the dose recommendation (e.g., administered the recommended dose within a threshold amount of time following generation of the dose recommendation) or did not comply with the dose recommendation (e.g., did not administer the recommended dose within a threshold amount of time following generation of the dose recommendation, administered a higher or lower dose than recommended by the dose recommendation, or administered a dose despite the dose recommendation being a recommendation to not administer a dose). The user's behavior may be tracked by system 10 and used to generate a compliance assessment report that the user can view (e.g., via the user interface of health management application 40 and/or the user interface of smartphone 30). The user compliance assessment report may demonstrate to the user the effects over a given period of time of non-compliance with a dose recommendation versus compliance with a dose recommendation to gradually effect a behavior change in the user toward improved compliance with dose recommendations.

While several aspects of the present disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A system comprising:
one or more processors; and
one or more processor-readable storage media storing instructions which, when executed by the one or more processors, cause performance of:
obtaining a recommendation for a dose of medicine to be delivered to a patient;
determining that the recommendation is outdated based on detecting one or more intervening events occurring after generation of the recommendation but before any delivery of medicine in accordance with the recommendation; and
implementing one or more safety features for avoiding delivery of the dose of medicine to the patient.

2. The system according to claim 1, wherein implementing the one or more safety features comprises causing presentation, via a user interface of a medicine delivery device, information indicating that the recommendation is unreliable.

3. The system according to claim 2, wherein the medicine delivery device is a medicine injection device.

4. The system according to claim 2, wherein the user interface comprises one or more light-emitting diodes (LEDs).

5. The system according to claim 2, wherein causing presentation of the information comprises causing one or more LEDs to illuminate a particular color.

6. The system according to claim 2, wherein the information indicates that the recommendation is unreliable due to the recommendation being outdated.

7. The system according to claim 2, wherein the information indicates that the recommendation is unreliable due to passage of a threshold period of time since communication between the medicine delivery device and a computing device.

8. The system according to claim 1, wherein implementing the one or more safety features comprises causing activation of a mechanical lock-out mechanism configured to prevent delivery of medicine from a medicine delivery device.

9. The system according to claim 1, wherein detecting the one or more intervening events comprises determining passage of a threshold period of time since generation of the recommendation.

10. The system according to claim 1, wherein detecting the one or more intervening events comprises obtaining a superseding recommendation for an updated dose of medicine to be delivered to the patient.

11. The system according to claim 1, wherein the one or more processor-readable storage media further store instructions which, when executed by the one or more processors, cause performance of:
after obtaining the recommendation, determining that the recommendation is up-to-date based on an absence of intervening events occurring after generation of the recommendation but before any delivery of medicine in accordance with the recommendation; and
causing presentation, via a user interface of a medicine delivery device, information indicating that the recommendation is reliable.

12. A processor-implemented method comprising:
obtaining a recommendation for a dose of medicine to be delivered to a patient;
determining that the recommendation is outdated based on detecting one or more intervening events occurring after generation of the recommendation but before any delivery of medicine in accordance with the recommendation; and
implementing one or more safety features for avoiding delivery of the dose of medicine to the patient.

13. The method according to claim 12, wherein implementing the one or more safety features comprises causing presentation, via a user interface of a medicine delivery device, information indicating that the recommendation is unreliable.

14. The method according to claim 13, wherein the user interface comprises one or more light-emitting diodes (LEDs), and wherein causing presentation of the information comprises causing one or more LEDs to illuminate a particular color.

15. The method according to claim 13, wherein the information indicates that the recommendation is unreliable due to the recommendation being outdated.

16. The method according to claim 13, wherein the information indicates that the recommendation is unreliable due to passage of a threshold period of time since communication between the medicine delivery device and a computing device.

17. The method according to claim 12, wherein implementing the one or more safety features comprises causing activation of a mechanical lock-out mechanism configured to prevent delivery of medicine from a medicine delivery device.

18. The method according to claim 12, wherein detecting the one or more intervening events comprises determining passage of a threshold period of time since generation of the recommendation.

19. The method according to claim 12, wherein detecting the one or more intervening events comprises obtaining a superseding recommendation for an updated dose of medicine to be delivered to the patient.

20. One or more non-transitory processor-readable storage media storing instructions which, when executed by one or more processors, cause performance of:
obtaining a recommendation for a dose of medicine to be delivered to a patient;
determining that the recommendation is outdated based on detecting one or more intervening events occurring after generation of the recommendation but before any delivery of medicine in accordance with the recommendation; and
implementing one or more safety features for avoiding delivery of the dose of medicine to the patient.
